(19) United States
(12) Patent Application Publication
(10) Pub. No.: US 2004/0204493 A1
(43) Pub. Date: Oct. 14, 2004

(54) Carbonic anhydrase inhibitors as drugs to eradicate Helicobacter pylori in the mammalian, including human, stomach

(76) Inventors: David R. Scott, Calabasas, CA (US); Elizabeth A. Marcus, Sepulveda, CA (US); George Sachs, Encino, CA (US)

Correspondence Address: GABOR L. SZEKERES, 8141 E. KAI SER BLVD., SUITE 112, ANAHEIM HILLS, CA, 92808, US

(21) Appl. No.: 10/817,626

(22) Filed: Apr. 2, 2004

Related U.S. Application Data
(60) Provisional application No. 60/462,451, filed on Apr. 11, 2003.

(51) Int. Cl. A61K 31/18

(52) U.S. Cl. 514/602

(57) ABSTRACT

Inhibitors of the alpha carbonic anhydrase inhibitors are administered to treat H. pylori infection in the mammalian, including human, stomach. The inhibitors are administered to mammals, including humans, in an amount that results in 4×10⁻⁴ to 4×10⁻⁹ Molar concentration of the drug in the stomach because this concentration range is lethal to the H. pylori bacteria.

### BACKGROUND OF THE INVENTION

1. Field of the Invention

The present invention is in the field of pharmaceutical compositions and methods for treating H. pylori infection in the mammalian, including human, stomach. More particularly, the present invention pertains to the use of inhibitors of the enzyme carbonic anhydrase in pharmaceutical compositions and methods to treat H. pylori infection in the mammalian, including human, stomach and thereby to prevent development of cancer of the stomach, and to prevent, ameliorate or cure ulcers of the stomach.

2. Description of Background Art

Helicobacter pylori is a bacterium that colonizes the human stomach. If left untreated H. pylori colonization results in a persistent, lifelong infection, (Blaser Helicobacter pylori and gastric diseases. BMJ 1998;316:1507-1510 and Blaser Epidemology and pathophysiology of Camplyobacter pylori infections. Rev Infec Dis 1990;12(suppl 1):S99-S106). It is predicted that without intervention, H. pylori infection will remain endemic in the US for at least another century (Rupnow et al. A dynamic transmission model for predicting trends in Helicobacter pylori and associated diseases in the United States. Emerg Infect Dis 2000; 6: 228-237.) Fifteen to twenty percent of patients with H. pylori infection develop peptic ulcers and 3% of infected patients develop gastric cancer. At least two forms of gastric cancer have been associated with H. pylori infection, adenocarcinoma and, less commonly, mucosa-associated lymphoid tissue (MALT) lymphoma (Blaser Helicobacter pylori and gastric diseases., supra). Eradication of H. pylori in patients with DU or gastric ulcer cures the disease and prevents relapse and reduces the risk of cancer (Uemura et al. Helicobacter pylori infection and the development of gastric cancer. N Engl J Med 2001;345:784-789., Effect of Helicobacter pylori eradication on subsequent development of cancer after endoscopic resection of early gastric cancer. Cancer Epidemiol Biomarkers Prev 1997;6:639-642). It is found that even with long term infection eradication results in a decreased incidence of gastric cancer (Akre et al. Risk for gastric cancer after antibiotic prophylaxis in patients undergoing hip replacement. Cancer Res. 2000;60:6376-6380).

Current H. pylori eradication therapy requires a proton pump inhibitor (PPI) and at least two antibiotics and is known as “triple therapy”. Standard eradication therapy requires treatment for 14 days and has a success rate of 80%. Compliance has been a factor in unsuccessful eradication since twice daily dosing of a PPI and 2 antibiotics for 14 days requires 84 tablets and conformity with before meal administration due to the mechanism of the PPIs (Penston et al. Eradication of Helicobacter pylori: an objective assessment of current therapies. Br J Clin Pharmacol 1997;43:223-243). Additionally, the efficacy of various triple therapy regimes is undermined by the development of antimicrobial resistance by H. pylori (Graham Antibiotic resistance in Helicobacter pylori: implications for therapy. Gastroenterology, 1998; 115: 1272-1277) and most likely contributes to development of antibiotic resistance of other important bacterial pathogens to certain antibacterial agents, such as clarithromycin and metronidazole (Megraud Resistance of Helicobacter pylori to antibiotics. Aliment Pharmacol Ther 1997;11(Suppl. 1):43-53; Meyer et al. Risk factors for Helicobacter pylori resistance in the United States: the Surveillance of H. pylori Anti; Dore et al., Effects of pretreatment antibiotic resistance to metronidazole and clarithromycin on outcome of Helicobacter pylori therapy: a meta-analytical approach. Dig Dis Sci 2000; 45:68-76.).

In earlier studies involving humans, acetazolamide, a compound which has carbonic anhydrase inhibitory activity, was found to inhibit both basal acid secretion and stimulated acid secretion. (see Puscas et al. anhydrase inhibitors in the treatment of gastric and duodenal ulcers. Arch Fr Mal App Dig 1976;65:577-83.) Acetazolamide reduced basal acid output (BAO) by 92.3% and histamine stimulated acid secretion (MAO) by 83.2%. Maximal acid inhibition with acetazolamide was observed after 3-5 days of treatment and normal acid secretion returned 3-5 following cessation of treatment. In one study of 118 gastric ulcer patients, orally administered acetazolamide abolished intragastric pain in 94% of the patients after 3-5 days (see Valean S, Vlaicu R, Ionescu I. Treatment of gastric ulcer with carbonic anhydrase inhibitors. Ann N Y Acad Sci 1984;429:597-600.)

Endoscopic evaluation after 15 and 30 days of treatment showed complete ulcer healing in 87% and 94% of the patients, respectively. Similar data were found in other studies (see Puscas et al. supra, Puscas I Treatment of gastroduodenal ulcers with carbonic acid inhibitors. Ann N Y Acad Sci 1984;429:587-591 and Erdei et al. Successful treatment of intractable gastric ulcers with acetazolamide. Acta Med Hung 1990;47:171-178). The high rate of ulcer healing after 30 days in the presence of carbonic anhydrase inhibitor is impressive but similar results are obtained with PPIs and H2RAs that are more effective inhibitors of acid secretion.

Cessation of acid inhibitory treatment without H. pylori eradication results in a return of symptoms in approximately 60% of patients within the first year after cessation of PPI or H2RA therapy. In contrast, 2 years after Diamox® treatment the relapse rate in patients treated with acetazolamide for 30 days was only 6.2% while that of an antacid treated control group was 43% (see Valean et al. supra). In another study, 3 week treatment with acetazolamide resulted in ulcer healing with a relapse rate of 11% (see Erdei et al. supra). This is reminiscent of modern data where H. pylori has been eradicated along with acid blockade to heal the ulcer and this effect was not recognized as being due to H. pylori eradication in any of the previous investigations and suggests that the efficacy of acetazolamide was not because of its inhibition of acid secretion but because H. pylori was eradicated. These studies have not been followed up in the modern era of proton pump inhibitors (PPIs) and triple therapy and have not been recognized as due to carbonic anhydrase inhibitory activity until the present invention.

It follows from the foregoing that pharmaceutical compositions and methods of treatment are missing in the prior art which would eradicate the H. pylori bacteria in the mammalian, primarily human, stomach without the complications, costs and risk factors associated with the current therapy. The present invention provides such pharmaceutical compositions and methods of treatment.

### SUMMARY OF THE INVENTION

In accordance with the invention, alpha carbonic anhydrase inhibitors are used to treat H. pylori infection in the mammalian, including human, stomach. The alpha carbonic anhydrase inhibitors are administered to mammals, including humans, in such amount that the concentration of the drug in the stomach is in the range of 4×10⁻⁴ to 4×10⁻⁹ Molar because this concentration range is lethal to the H. pylori bacteria. A likely daily oral dose range of the alpha carbonic anhydrase inhibitors when administered to mammals, particularly humans, in need of such administration, is 5 to 30 mg of the drug per kg body weight of the patient. Administering the drug in these doses is likely to completely eradicate H. pylori in the stomach and thus not only treat but cure peptic ulcer disease and prevent gastric cancer.

### BRIEF DESCRIPTION OF THE DRAWING FIGURE

FIG. 1 shows incubation in acidic medium in the presence of urea and a fluorescent pH sensitive dye of normal H. pylori bacteria (part A), carbonic anhydrase gene deficient H. pylori bacteria (part B) and normal H. pylori bacteria in the presence of Diamox® (part C).

FIG. 2 is a diagrammatic representation of the action of urease and carbonic anhydrase enzymes in the cytoplasm and periplasm, respectively, and of the diffusion of NH₃ and CO₂ gases.

### DETAILED DESCRIPTION OF THE INVENTION

The biological and physiological mechanism of action for the working of α-carbonic anhydrase inhibitors to eradicate the H. pylori bacteria from the mammalian, including human stomach, as presently understood by the inventors based on experiments involving the bacteria, is as follows.

H. pylori, like E. coli, is a neutralophile, an organism that requires a near neutral pH for growth. Because the medium of the mammalian stomach is highly acidic H. pylori has evolved acid resistance mechanisms to combat gastric acidity to uniquely colonize the stomach. A major adaptation to acid is the constitutive production of large amounts of intra-bacterial urease enzyme (Mobley et al. Molecular biology of microbial ureases. Microbiol Rev 1995;59:451-480). This urease activity is crucial for bacterial colonization and survival in the harsh acidic environment of the stomach. Activation of urease at acidic pH occurs through the opening of the proton-gated urea channel (UreI) that allows rapid entry of urea into the cytoplasm where it hydrolyzed by the intra-bacterial urease. This results in intra-bacterial production of ammonia (NH₃)and carbon dioxide (CO₂). It was previously thought that the ammonia produced by urea hydrolysis is able to raise the pH of the periplasm allowing normal cellular functions of H. pylori bacteria. However, recent experiments have shown that the presence of carbonic anhydrase activity is also essential for the periplasmic buffering action induced by the acid activation of UreI (Scott et al. Expression of the Helicobacter pylori ureI Gene is Required for Acidic pH Activation of Cytoplasmic Urease. Infec Immun 2000;68(2):470-477).

Without necessarily being bound by theory, the function of carbonic anhydrase along with the products of urea hydrolysis is likely to be an NH₃ trapping mechanism due to the rapid formation of HCO₃⁻from the CO₂ that also diffuses into the periplasm from inside the bacterium to generate NH₄HCO₃ as buffer in the periplasm. The foregoing is shown below by the equations that summarize the relationship between urease activity and periplasmic carbonic anhydrase activity.

The urease enzyme catalyzes the reaction:

CO(NH₂)₂+H₂0→NH₃+NH₂COOH and this is followed rapidly by the spontaneous reactions:

NH₂COOH+H₂0→NH₃+H₂C0₃

H₂C0₃→HCO₃⁻+H⁺

In the presence of cytoplasmic carbonic anhydrase the bicarbonate can be rapidly converted to CO₂ gas by the reaction

HCO₃⁻→CO₂+OH⁻with then the net cytoplasmic production of two molecules of ammonia and one molecule of carbon dioxide gas (2NH₃+CO₂) in the cytoplasm. Both gases can rapidly cross membranes, into the periplasm and into the medium. The CO₂ is converted to HCO₃⁻+H⁺ by the periplasmic α-carbonic anhydrase. The periplasmic HCO₃⁻ along with one of the effluxed NH₃ form NH₄ HCO₃⁻ which buffers the periplasmic pH at 6.1. The other ammonia absorbs medium protons to form NH₄⁺. The periplasmic reactions are therefore:

CO₂+H₂O+2NH₃+H⁺_{medium} →2NH₄⁺+HCO₃⁻and both components are essential for acid survival of the organism.

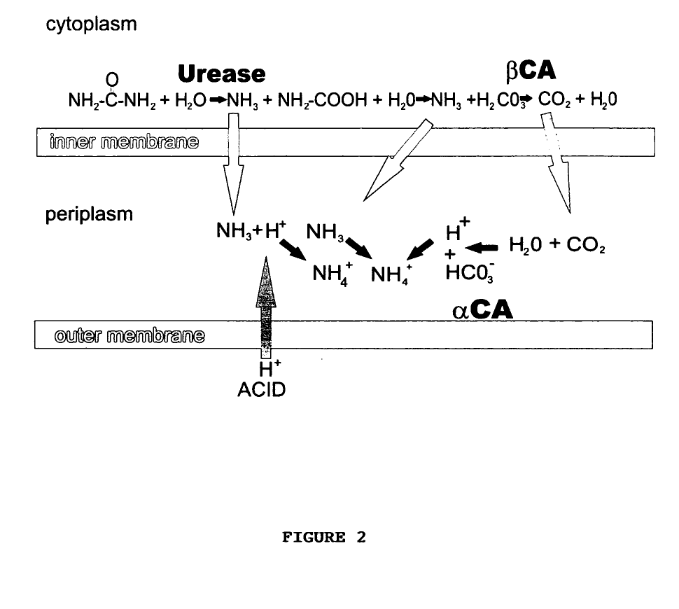

A model of the requirement for both urease and carbonic anhydrase is shown in appended FIG. 2. Urea hydrolysis in the cytoplasm generates 2NH₃ and CO₂. These gases diffuse into the periplasm. There the periplasmic carbonic anhydrase catalyses the formation of HCO₃⁻+H⁺. This then forms NH₄⁺ generating the buffer NH₄⁺HCO₃⁻. The other NH₃ neutralizes acid entering from the medium.

In animal experiments ureI deletion mutants of the bacteria were able to infect the gerbil stomach only when acid secretion was inhibited by a PPI and the bacteria were eradicated when acid secretion was restored showing that the gastric environment was routinely acidic and lethal to the microbe in the absence of periplasmic buffering. (see Mollenhauer-Rektorschek et al., Res Microbiol. 153:659-666, 2002).

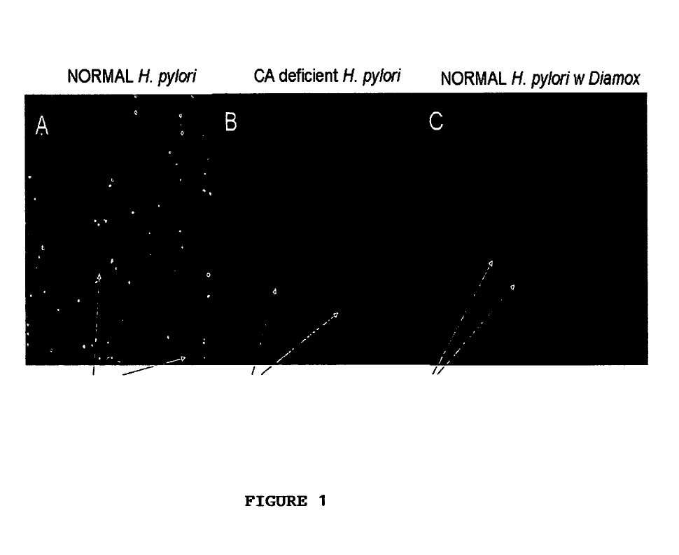

Consistent with the need for carbonic anhydrase activity for acid survival, micro-array analysis of pH sensitive gene regulation showed that periplasmic α-carbonic anhydrase was up-regulated more than three fold even under mildly acidic conditions (pH 4.5) in the presence of urea (Wen et al. Acid adaptive genes of Helicobacter pylori. Infect Immun 2003 71(10):5921-39). H. pylori bacteria which could not produce α-carbonic anhydrase gene (deletion mutants) were unable to survive in acid and were incapable of raising their periplasmic pH even with wildtype urease activity levels. In vitro treatment of wildtype H. pylori with the carbonic anhydrase inhibitor, acetazolamide (Diamox®) also inhibited the bacterial ability to survive in acid or buffer its periplasm without affecting urease activity, again suggesting to the present inventors that α-carbonic anhydrase plays a crucial role in the acid survival of H. pylori. Particularly striking is the demonstration that while the normal organism can buffer its periplasm in acid with the addition of urea, this periplasmic buffering is absent in either the organism in which carbonic anhydrase has been genetically deleted or when Diamox® is present in the solution. This is illustrated in FIG. 1 of the appended drawings.

Specifically, H. pylori was incubated in acid along with a fluorescent pH sensitive dye, BCECF, (bis-carboxy ethylcarboxy fluorescein) and then urea was added at 5 mM which is substantially the gastric juice concentration. In part A of the figure the increase in periplasmic pH is shown by the increased fluorescence of the dye indicating viable H. pylori bacteria. Part B of the figure shows incubation of bacteria that have their periplasmic carbonic anhydrase gene deleted, there is no periplasmic buffering of pH detected. Part C of the figure shows incubation of normal H. pylori but Diamox® has been added to the medium, there is no periplasmic buffering of pH detected.

Therefore, it is now understood in accordance with the present invention that it is the combination of urease activation, expression of UreI and the periplasmic carbonic anhydrase that allows gastric colonization by H. pylori. Hence there is more than a 3 log order of magnitude loss of survival of H. pylori in acid, either with genetic removal of UreI or carbonic anhydrase or in the presence of Diamox®. Thus, targeting the acid biology of this pathogen provides specific H. pylori therapy. It is recognized in accordance with the present invention that within these three targets, a safe drug is only available for carbonic anhydrase.

Except for the drug known as Diamox®, any potent carbonic anhydrase inhibitor can be used in accordance with the present invention for the preparation of formulations suitable for oral administration to mammals, including humans, infected by H. pylori bacteria. Diamox® is not included within the scope of the invention because of the side effects caused by Diamox® in humans.

The most potent CA inhibitors belong to a group of compounds known as sulfonamides, and these serve as non-limiting examples for the pharmaceutical compositions and methods of treatment of the present invention. The potent sulfonamides include the clinically used acetazolamide, dorzolamide and brinzolamide.

Accordingly, carbonic anhydrase inhibitor drugs, including the above-mentioned sulfonamides, such as acetazolamide, dorzolamide, and brinzolamide, in the concentration range of 4×10⁻⁴ to 4×10⁻⁹ M will be lethal to H. pylori in the mammalian, including human, stomach when given in the presence of acid secretion. The effective dose can be arrived at through routine experimentation which is commonly performed by health care professionals for the treatment of individuals.

Keeping the foregoing in mind, likely daily dose range of the carbonic anhydrase inhibitors when administered to mammals, particularly humans, in need of such administration is 5 to 30 mg of the drug per kg body weight of the patient. The drug is to be administered orally in a tablet, capsule, soft-gel capsule or in any other form suitable for oral administration. Its formulation is likely to include such pharmaceutically acceptable excipients which are known in the art. The preparation of oral formulations is so well known in the art that a detailed description of preparing oral dosage forms for the carbonic anhydrase inhibitors in accordance with the present invention is not considered necessary. Preferably the daily dose is administered to human patients in two portions, one preferably in the morning and one approximately at bedtime. Administration of one or more carbonic anhydrase inhibitors in this manner to mammals, including humans, is expected to eradicate H. pylori in the stomach, and thus not only treat but cure peptic ulcer disease and prevent gastric cancer.

What is claimed is:

1. A process of administering to a mammal in need of such administration, for the purpose of eradicating H. pylori bacteria in the stomach of the mammal, a carbonic anhydrase inhibitor drug.

2. A process in accordance with claim 1 wherein the mammal is a human being.

3. A process in accordance with claim 2 where the drug is a sulfonamide.

4. A process in accordance with claim 3 where the drug is selected from the group consisting of acetazolamide, dorzolamide and brinzolamide.

5. A process in accordance with claim 4 where the drug is acetazolamide.

6. A process in accordance with claim 1 where the drug is administered in a daily dose that results in a 4×10⁻⁴ to 4×10⁻⁹ M concentration of the drug in the mammal's stomach.

7. A process in accordance with claim 6 wherein the mammal is a human being.

8. A process in accordance with claim 1 where a daily dose of 5 mg to 30 mg per kg body weight of the mammal is administered.

9. A process in accordance with claim 8 wherein the mammal is a human being.

10. A process of administering to a human in need of such administration, for the purpose of eradicating H. pylori bacteria in the human's stomach a carbonic anhydrase inhibitor drug in a daily dose that results in a 4×10⁻⁴ to 4×10⁻⁹ M concentration of the drug in the human's stomach.

11. A process in accordance with claim 10 where the drug is a sulfonamide.

12. A process in accordance with claim 11 where the drug is selected from the group consisting of acetazolamide, dorzolamide and brinzolamide.

13. A process in accordance with claim 12 where the drug is acetazolamide.

14. A process in accordance with claim 10 where a daily dose of 5 mg to 30 mg per kg body weight of the human is administered.

15. A process in accordance with claim 14 where the drug is a sulfonamide.

16. A process in accordance with claim 15 where the drug is selected from the group consisting of acetazolamide, dorzolamide and brinzolamide.

17. A process in accordance with claim 16 where the drug is acetazolamide.

18. A pharmaceutical composition comprising a carbonic anhydrase inhibitor drug in a quantity that with administration of a daily dose results in a 4×10⁻⁴ to 4×10⁻⁹ M concentration of the carbonic anhydrase inhibitor drug in the stomach of a mammal, the composition being for the purpose of eradicating H. pylori bacteria in the stomach of the mammal.

19. A pharmaceutical composition in accordance with claim 18 where the drug is a sulfonamide.

20. A pharmaceutical composition in accordance with claim 19 where the drug is selected from the group consisting of acetazolamide, dorzolamide and brinzolamide.

21. A pharmaceutical composition in accordance with claim 20 where the drug is acetazolamide.
